(19) United States
(12) Patent Application Publication
(10) Pub. No.: US 2024/0350819 A1
(43) Pub. Date: Oct. 24, 2024

(54) Implantable System and Method for Providing Anti-Tachycardia and/or Shock Therapy

(71) Applicant: BIOTRONIK SE & Co. KG, Berlin (DE)

(72) Inventors: Thomas DOERR, Berlin (DE); Ingo WEISS, Berlin (DE)

(73) Assignee: BIOTRONIK SE & Co. KG, Berlin (DE)

(21) Appl. No.: 18/685,285

(22) PCT Filed: Aug. 19, 2022
(86) PCT No.: PCT/EP2022/073204

§ 371 Date: Feb. 21, 2024

(30) Foreign Application Priority Data
Sep. 6, 2021 (EP) 21194956.5

(51) Int. Cl. A61N 1/39; A61N 1/362; A61N 1/365; A61N 1/372; A61N 1/375

(52) U.S. Cl. CPC A61N 1/39622; A61N 1/3621; A61N 1/365; A61N 1/37223; A61N 1/37235; A61N 1/3756

(57) ABSTRACT

An implantable system for providing anti-tachycardia and/or shock therapy, comprising an implantable pacing device, in particular an implantable leadless pacemaker, and an implantable cardioverter defibrillator, in particular a non-transvenous implantable cardioverter defibrillator, wherein the implantable pacing device is configured to detect a tachycardia and to provide anti-tachycardia pacing, wherein the implantable cardioverter defibrillator is further configured to signal an availability and/or unavailability of defibrillation to the implantable pacing device, and wherein the implantable pacing device is configured, in response to the signal of the implantable cardioverter defibrillator to enable and/or disable anti-tachycardia pacing. The invention further relates to a computer implemented method for providing anti-tachycardia and/or shock therapy, a computer program and a computer readable data carrier.

### CROSS-REFERENCE TO RELATED APPLICATIONS

This application is the United States National Phase under 35 U.S.C. § 371 of PCT International Patent Application No. PCT/EP2022/073204, filed on Aug. 19, 2022, which claims the benefit of European Patent Application No. 21194956.5, filed on Sep. 6, 2021, the disclosures of which are hereby incorporated by reference herein in their entireties.

### TECHNICAL FIELD

The present invention relates to an implantable system for providing anti-tachycardia and/or shock therapy. Furthermore, the present invention relates to a computer implemented method for providing anti-tachycardia and/or shock therapy.

### BACKGROUND

Such an implantable system also known as cardiac rhythm management system (CRMS) can be used for electric stimulation therapy of cardiac arrhythmia. Said cardiac rhythm management system comprises at least one first implantable stimulation device, for example, an implantable leadless pacemaker (iLP), and at least one second implantable stimulation device, for example, a subcutaneous implantable cardioverter defibrillator (S-ICD), wherein the at least one first implantable stimulation device comprises a first detection unit adapted to detect a patient's cardiac rhythm and a first processor adapted to analyze the detected patient's cardiac rhythm and to deliver signals for a first anti-tachycardia pacing therapy, wherein the at least one second implantable stimulation device comprises a second detection unit adapted to detect the patient's cardiac rhythm and a second processor adapted to analyze the detected patient's cardiac rhythm and to deliver signals for shock therapy.

Implantable stimulation devices such as implantable cardiac pacemakers or implantable leadless pacemakers are well known medical devices that allow stimulation of the heart of a patient. In general, those medical devices are battery operated and a stimulation component is directly implanted into the heart's ventricle or atrium. Implantable cardiac pacemakers have at least an elongated stimulation lead which reaches from the device housing into a heart chamber where it is anchored. Implantable leadless pacemakers are miniaturized pacing devices which are entirely implanted into the heart chamber.

Implantable stimulation devices with a defibrillation function are known in the art, as for instance implantable cardioverter-defibrillators (ICDs) or non-transvenous implantable cardioverter defibrillators, for example, subcutaneous implantable cardioverter-defibrillators (S-ICDs). Such devices typically comprise of a device housing and at least one elongated stimulation lead which extends from the housing. The housing of an ICD is typically implanted in a skin pocket below the clavicle, wherein the stimulation lead reaches into the ventricle of the heart where it is fixed. The housing and stimulation lead of an non-transvenous implantable cardioverter defibrillator are implanted under the skin (i.e., subcutaneously), in a way that a shock vector that runs through the cardiac ventricles is created between the stimulation electrode(s) of the lead and the non-transvenous implantable cardioverter defibrillator housing.

The medical device is chosen according to the patient's cardiac condition, i.e., the required cardiac therapy. Implantable pacemakers or implantable leadless pacemakers are used for patients who suffer from a bradycardia, that is if a heart that beats too slow to fulfil the physiological needs of the patient. The implantable pacemaker or implantable leadless pacemaker applies electrical stimulation to the heart in order to generate a physiologically appropriate heartrate.

ICDs are used for patients who suffer from ventricular tachycardia and fibrillations. The ICD is able to apply anti-tachycardia pacing (ATP) therapy (i.e., pacing the heart with a faster stimulation rate than the tachycardia rate) to terminate a tachycardia, or a shock therapy (i.e., high energetic electric shock which is applied to the ventricles to terminate the tachycardia to bring back the heart to a physiological rhythm) if the tachycardia persists after ATP attempts.

non-transvenous implantable cardioverter defibrillators are configured to deliver a shock therapy, but no pacing therapy or ATP therapy. That is due to the distance between stimulation lead and the cardiac chambers, so that a low energetic stimulation pulse could not be delivered effectively to a cardiac pacing site.

An iLP may deliver pacing therapy and ATP, but no shock therapy. Due to the highly restricted device size, it has a small battery capacity and lack of space for charging capacitors required for providing a shock therapy.

Moreover, implantable leads pose a risk to the patient and can therefore be a problem. The lead is an elongated insulated electrode wire which reaches from the device housing into the venous system of the heart where it is anchored in the ventricle. It undergoes different forces and movements with every beat of the heart, which can result in lead dislodgement, insulation failures and lead breach. That problem does not occur with non-transvenous implantable cardioverter defibrillators and implantable leadless pacemakers, because these devices have no intracardiac elongated lead. Especially for patients who have no adequate vascular access or are at high risk for infection, no elongated leads can be implanted inside the heart.

However, there are circumstances in which a patient suffers from various cardiac arrhythmias that require different cardiac therapies. In such cases, a CRMS may be implanted comprising at least two medical devices or units.

Furthermore, there exist cardiac arrhythmias for which different therapies are suitable and one treatment is more favorable, e.g., more comfortable, for the patient. Further, some therapies may cause another arrhythmia, so that an additional therapy is required in order to stop this arrhythmia. In practice, ventricular tachycardia, for example, may be treated using ATP therapy or shock therapy, wherein shock therapy is often uncomfortable for patients as the shocks are emitted unexpectedly and may be painful. In addition, shock therapies cause a considerable decrease in the longevity of the battery. Nevertheless, shock therapy is inevitable if a ventricular tachycardia leads to ventricular fibrillation as ATP therapy is not suitable to treat fibrillations.

For instance, a patient who has a contraindication for intracardiac elongated leads and who suffers from ventricular tachycardia requires pacing therapy, ATP and shock therapy. In such case, a CRMS may be implanted comprising at least a first implantable stimulation device and a second implantable stimulation device, wherein the first implantable stimulation device may be an implantable leadless pacemaker, and the second device a non-transvenous implantable cardioverter defibrillator.

According to another example, if a patient who has a contraindication for intracardiac elongated leads and who requires pacing therapy and/or ATP, in the ventricle (or at the HIS bundle) and in the atrium, a CRMS may be implanted comprising at least a first implantable stimulation device and a second implantable stimulation device, wherein the first implantable stimulation device may be a first implantable leadless pacemaker, and the second device a second implantable leadless pacemaker.

Cardiac rhythm management systems comprising multiple treatment therapies are, for example, provided by a combination of S-ICD and iLP as disclosed in the prior art documents U.S. Publication No. 2019/0160285 A1 and U.S. Pat. No. 10,265,534 B2. The coordination of such systems is obligatory in order to provide proper treatment as the therapies may be ineffective if they are applied simultaneously.

Furthermore, U.S. Publication No. 2016/0008615 A1 relates to a medical device system for delivering electrical stimulation therapy to a heart of a patient, the system comprising a leadless cardiac pacemaker LCP implanted within a heart of a patient and configured to determine occurrences of cardiac arrhythmias, a medical device configured to determine occurrences of cardiac arrhythmias and to deliver defibrillation shock therapy to the patient, wherein the LCP and the medical device are spaced from one another and communicatively coupled, and wherein after the LCP determines an occurrence of a cardiac arrhythmia, the LCP is configured to modify the defibrillation shock therapy of the medical device.

In addition, U.S. Publication No. 2018/0243578 A1 discloses an ambulatory medical device comprising at least one therapy electrode configured to couple externally to a skin of a patient and to provide one or more transthoracic therapeutic stimulation pulses to a heart of the patient, at least one sensing electrode configured to couple externally to the skin of the patient and to acquire electrocardiogram (ECG) signals from the patient, and at least one processor coupled to the at least one therapy electrode and the at least one sensing electrode and configured to process the ECG signals from the patient to detect a tachycardia condition in the heart of the patient, determine, in response to detecting the tachycardia condition, whether an implanted pacemaker restores the heart of the patient to a normal condition within a predetermined period, and provide the one or more transthoracic therapeutic stimulation pulses to the heart of the patient in response to determining that the implanted pacemaker failed to restore the heart of the patient to the normal condition within the predetermined period.

The above-mentioned cardiac rhythm management systems have in common that the provision of anti-tachycardia pacing (ATP), in the event of an ATP-induced acceleration of ventricular tachycardia needs to be followed by shock therapy performed by the implantable cardioverter defibrillator (ICD).

If, however, due to a fault condition or battery depletion the implantable cardioverter defibrillator (ICD) is unable to provide shock therapy, the anti-tachycardia pacing (ATP) may potentially lead to ATP-induced acceleration of ventricular tachycardia.

The present disclosure is directed toward overcoming one or more of the above-mentioned problems, though not necessarily limited to embodiments that do.

### SUMMARY

It is therefore an object of the present invention to provide an improved implantable system for providing anti-tachycardia and/or shock therapy capable of reducing the risk of ATP-induced acceleration of ventricular tachycardia.

At least the object is solved by an implantable system for providing anti-tachycardia and/or shock therapy having the features of claim 1.

Furthermore, at least the object is solved by a computer implemented method for providing anti-tachycardia and/or shock therapy having the features of claim 13.

Moreover, at least the object is solved by a computer program having the features of claim 14 and a computer-readable data carrier having the features of claim 15.

Further developments and advantageous embodiments are defined in the dependent claims.

The present invention provides an implantable system for providing anti-tachycardia and/or shock therapy, comprising an implantable pacing device, in particular an implantable leadless pacemaker, and an implantable cardioverter defibrillator, in particular a non-transvenous implantable cardioverter defibrillator, wherein the implantable pacing device is configured to detect a tachycardia and to provide anti-tachycardia pacing, wherein the implantable cardioverter defibrillator is further configured to signal an availability and/or unavailability of defibrillation to the implantable pacing device, and wherein the implantable pacing device is configured, in response to the signal of the implantable cardioverter defibrillator to enable and/or disable anti-tachycardia pacing.

Furthermore, the present invention provides a computer implemented method for providing anti-tachycardia and/or shock therapy.

The method comprises providing an implantable system for providing anti-tachycardia and/or shock therapy, comprising an implantable pacing device, in particular an implantable leadless pacemaker, and an implantable cardioverter defibrillator, in particular a non-transvenous implantable cardioverter defibrillator.

Furthermore, the method comprises detecting a tachycardia and providing anti-tachycardia pacing, by means of the implantable pacing device. In addition, the method comprises signaling an availability and/or unavailability of defibrillation to the implantable pacing device by means of the implantable cardioverter defibrillator.

The method moreover comprises enabling and/or disabling anti-tachycardia pacing by means of the implantable pacing device, in response to the signal of the implantable cardioverter defibrillator. Furthermore, the present invention provides a computer program with program code to perform the method of the present invention when the computer program is executed on a computer.

Moreover, the present invention provides a computer-readable data carrier containing program code of a computer program for performing the method of the present invention when the computer program is executed on a computer.

An idea of the present invention is to provide an implantable system for cardiac anti-tachycardia therapy comprising an implantable pacing device, and a non-transvenous ICD, wherein the implantable pacing device is capable of autonomously detecting tachycardia and delivering anti-tachycardia pacing according to the present invention only whenever the ICD is ready for use. The present invention thus provides a safe combination of a modular therapy system for tachycardia treatment using ATP and shock even in the event that the defibrillation function is inactive.

According to an aspect of the present invention, the implantable pacing device is configured to stimulate at least one ventricle of a human or animal heart, and wherein the implantable pacing device comprises a configurable tachyarrhythmia detection unit configured to detect a tachycardia and further comprises an anti-tachycardia pacing timing unit configured to deliver an anti-tachycardia pacing sequence in response to tachycardia detection.

An ATP sequence usually comprises 5-8 pacing pulses. These are delivered slightly faster than the actual detected tachycardia. The cycle length of the tachycardia is measured in the detection unit and then the timing of these 5-8 pulses is calculated in the timing unit, which have, for example, 80% of the cycle length of the tachycardia and thus effectively overstimulate said tachycardia.

According to a further aspect of the present invention, the implantable pacing device comprises a receiver unit configured to receive a signal indicating an availability or unavailability of defibrillation of the implantable cardioverter defibrillator, and wherein the implantable pacing device comprises a control unit configured to enable anti-tachycardia pacing if it is detected by the receiver unit that defibrillation by the implantable cardioverter defibrillator is available and to disable anti-tachycardia pacing if it is detected by the receiver unit that defibrillation by the implantable cardioverter defibrillator is unavailable. Thus, a safe provision of ATP therapy can be advantageously provided.

According to a further aspect of the present invention, the receiver unit is configured to evaluate the signal indicating the availability or unavailability of defibrillation by the implantable cardioverter defibrillator, and wherein the implantable pacing device is configured to perform a painless electrode impedance measurement of the implantable cardioverter defibrillator if it is determined that defibrillation by the implantable cardioverter defibrillator is available.

The information of the availability of defibrillation by the implantable cardioverter defibrillator can thus be advantageously used to decide whether or not the electrode impedance measurement of the implantable cardioverter defibrillator can be carried out.

According to a further aspect of the present invention, the implantable cardioverter defibrillator comprises a fault detection device configured to detect the unavailability of defibrillation, in particular due to a battery exhaustion and/or fault conditions of the implantable cardioverter defibrillator. Thus, the cause of the unavailability of defibrillation can advantageously be detected.

According to a further aspect of the present invention, the implantable cardioverter defibrillator is configured to signal the implantable pacing device by means of intra-body-communication, in particular by means of electrical pulses, communication and/or radio signals. Therefore, advantageously no cable-based connection needs to be present between the implantable pacing device and the implantable cardioverter defibrillator.

According to a further aspect of the present invention, the implantable pacing device prompts the cardioverter defibrillator to signal the availability of defibrillation by means of intra-body-communication before delivering anti-tachycardia pacing. This advantageously provides an additional layer of security by prompting the cardioverter defibrillator before delivering anti-tachycardia pacing.

According to a further aspect of the present invention, an external device, in particular a programmer and/or a wireless communication device, connected to the cardioverter defibrillator is configured to signal the availability and/or unavailability of defibrillation to the implantable pacing device. This advantageously provides a redundant hardware structure due to the fact that said function is performed by an external device but may also be provided by the cardioverter defibrillator.

According to a further aspect of the present invention, the cardioverter defibrillator is configured to communicate a remaining operating time to the implantable pacing device, and wherein the implantable pacing device is configured to deactivate an anti-tachycardia pacing function after expiry, at expiry or a predetermined time period before expiry of an expected defibrillator operating time. The information of the expected remaining operating time of the cardioverter defibrillator can thus advantageously be used to determine the best point in time to deactivate the anti-tachycardia pacing function of the implantable pacing device.

According to a further aspect of the present invention, the cardioverter defibrillator is configured to signal the availability of defibrillation by performing a painless impedance measurement of its electrode system only when the cardioverter defibrillator is in an activated state. This advantageously provides an additional way of signaling availability of defibrillation to the implantable pacing device.

According to a further aspect of the present invention, the cardioverter defibrillator is configured to signal the availability of defibrillation by emitting a signaling pattern only when the cardioverter defibrillator is in an activated state and the cardioverter defibrillator detects tachycardia. Thus, safe operation of the implantable system can be achieved.

According to a further aspect of the present invention, the cardioverter defibrillator comprises a communication unit for intrabody communication configured to signal the availability of defibrillation to the implantable pacing device. Said communication unit thus advantageously enables safe delivery of an anti-tachycardia pacing sequence in response to tachycardia detection.

The herein described features of the implantable system for providing anti-tachycardia and/or shock therapy are also disclosed for the computer implemented method for providing anti-tachycardia and/or shock therapy and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

For a more complete understanding of the present invention and advantages thereof, reference is now made to the following description taken in conjunction with the accompanying drawings. The present invention is explained in more detail below using exemplary embodiments, which are specified in the schematic figures of the drawings, in which:

FIG. 1 shows a schematic view of an implantable system for providing anti-tachycardia and/or shock therapy according to a preferred embodiment of the present invention:

FIG. 2a shows a schematic view of a therapy sequence of an implantable system for providing anti-tachycardia and/or shock therapy which does not form part of the present invention;

FIG. 2b shows a schematic view of a therapy sequence of the implantable system for providing anti-tachycardia and/or shock therapy according to the preferred embodiment of the present invention; and

FIG. 3 shows a flowchart of a computer implemented method for providing anti-tachycardia and/or shock therapy according to the preferred embodiment of the present invention.

### DETAILED DESCRIPTION

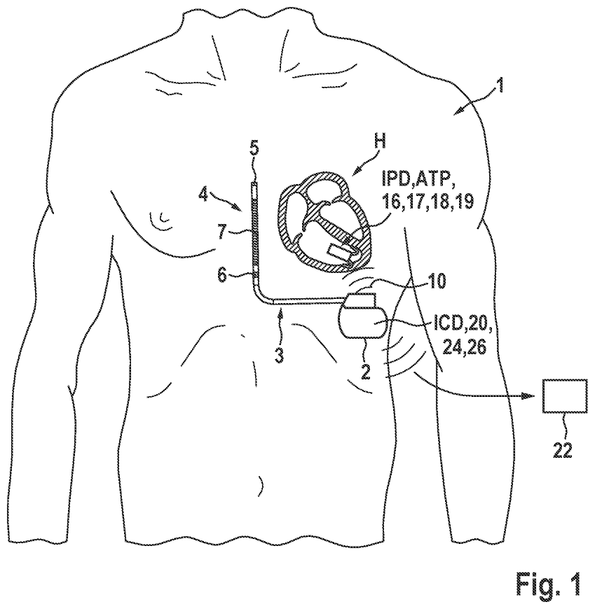

The implantable system 1 shown in FIG. 1 for providing anti-tachycardia and/or shock therapy comprises an implantable pacing device IPD, in particular an implantable leadless pacemaker, and an implantable cardioverter defibrillator ICD, in particular a non-transvenous implantable cardioverter defibrillator.

The implantable pacing device IPD comprises an active housing 2, an electrode lead 3 implanted subcutaneously along the sternum 4 and having two sensing poles 5, 6 and a shock coil 7.

The implantable pacing device IPD is configured to detect a tachycardia and to provide anti-tachycardia pacing ATP. Furthermore, the implantable cardioverter defibrillator ICD is configured to signal 10 an availability and/or unavailability of defibrillation to the implantable pacing device IPD. Moreover, the implantable pacing device IPD is configured, in response to the signal 10 of the implantable cardioverter defibrillator ICD to enable or disable anti-tachycardia pacing ATP.

The implantable pacing device IPD is configured to stimulate at least one ventricle of a human or animal heart H. Furthermore, the implantable pacing device IPD comprises a configurable tachyarrhythmia detection unit 16 configured to detect a tachycardia and further comprises an anti-tachycardia pacing timing unit 18 configured to deliver an anti-tachycardia pacing ATP sequence in response to tachycardia detection.

The implantable pacing device IPD comprises a receiver unit 17 configured to receive a signal indicating an availability or unavailability of defibrillation of the implantable cardioverter defibrillator ICD. In addition, the implantable pacing device IPD comprises a control unit 19 configured to enable anti-tachycardia pacing ATP if it is detected by the receiver unit 17 that defibrillation by the implantable cardioverter defibrillator ICD is available and to disable anti-tachycardia pacing ATP if it is detected by the receiver unit 17 that defibrillation by the implantable cardioverter defibrillator ICD is unavailable.

The receiver unit 17 is configured to evaluate the signal indicating the availability or unavailability of defibrillation by the implantable cardioverter defibrillator ICD. Furthermore, the implantable pacing device IPD is configured to perform a painless electrode impedance measurement of the implantable cardioverter defibrillator ICD if it is determined that defibrillation by the implantable cardioverter defibrillator ICD is available.

Moreover, the implantable cardioverter defibrillator ICD comprises a fault detection device configured to detect the unavailability of defibrillation, in particular due to a battery exhaustion and/or fault conditions of the implantable cardioverter defibrillator ICD. In addition, the implantable cardioverter defibrillator ICD is configured to signal 10 the implantable pacing device IPD by means of intra-body-communication, in particular by means of electrical pulses, communication and/or radio signals.

The implantable pacing device IPD prompts the cardioverter defibrillator ICD to signal the availability of defibrillation by means of intra-body-communication before delivering anti-tachycardia pacing ATP.

An external device 22, in particular a programmer and/or a wireless communication device, connected to the cardioverter defibrillator ICD is configured to signal the availability and/or unavailability of defibrillation to the implantable pacing device IPD.

The cardioverter defibrillator ICD is further configured to communicate a remaining operating time to the implantable pacing device IPD. Moreover, the implantable pacing device IPD is configured to deactivate an anti-tachycardia pacing ATP function after expiry, at expiry or a predetermined time period before expiry of an expected defibrillator operating time.

In addition, the cardioverter defibrillator ICD is configured to signal 10 the availability of defibrillation by performing a painless impedance measurement 24 of its electrode system only when the cardioverter defibrillator ICD is in an activated state.

The cardioverter defibrillator ICD is further configured to signal 10 the availability of defibrillation by emitting a signaling pattern only when the cardioverter defibrillator ICD is in an activated state and the cardioverter defibrillator ICD detects tachycardia.

Moreover, the cardioverter defibrillator ICD comprises a communication unit 26 for intrabody communication configured to signal 10 the availability of defibrillation to the implantable pacing device IPD.

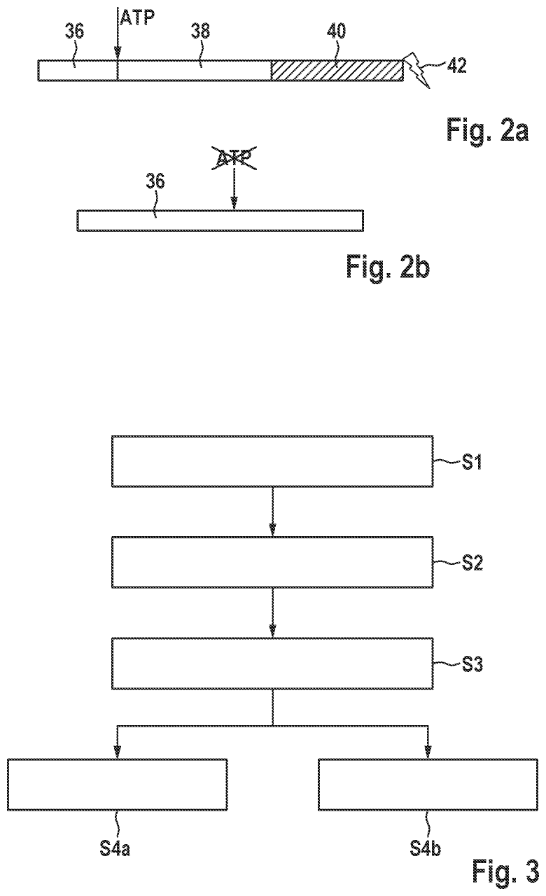

FIG. 2a shows a schematic view of a therapy sequence of an implantable system for providing anti-tachycardia and/or shock therapy which does not form part of the present invention.

A typical therapy sequence of the modular therapy system up to ICD shock delivery is shown. First, tachycardia detection 36 occurs-in parallel in the implantable pacing device IPD and the implantable cardioverter defibrillator ICD.

Following tachycardia detection, the implantable pacing device IPD delivers ATP therapy, with the implantable cardioverter defibrillator ICD initially withholding the initiation of shock therapy through a programmed therapy delay.

In the sequence outlined here, ATP therapy is not effective but accelerates the ventricular rhythm into life-threatening ventricular fibrillation 38.

Here, the activated implantable cardioverter defibrillator ICD detects the ventricular fibrillation and initiates a charging process for the shock capacitors 40 and delivers the life-saving defibrillation shock 42 after the charging process is complete.

FIG. 2b shows a schematic view of a therapy sequence of the implantable system 1 for providing anti-tachycardia and/or shock therapy according to the preferred embodiment of the present invention.

In particular FIG. 2b illustrates the situation in which the implantable cardioverter defibrillator ICD is disabled, e.g., by a user, by a fault condition or by battery depletion.

Here, ventricular tachycardia 36 is also detected by the implantable pacing device IPD. However, the pacemaker has been signaled 10 by the implantable cardioverter defibrillator ICD that a defibrillation function of the implantable cardioverter defibrillator ICD is deactivated and the implantable pacing device IPD consequently suppresses the programmed anti-tachycardia pacing ATP. This also precludes anti-tachycardia pacing ATP therapy from accelerating the tachycardia 36 and thus prevents causing a life-threatening condition.

FIG. 3 shows a flowchart of a computer implemented method for providing anti-tachycardia and/or shock therapy according to the preferred embodiment of the present invention.

The method comprises providing S1 an implantable system 1 for providing anti-tachycardia and/or shock therapy, comprising an implantable pacing device IPD, in particular an implantable leadless pacemaker, and an implantable cardioverter defibrillator ICD, in particular a non-transvenous implantable cardioverter defibrillator.

Furthermore, the method comprises detecting S2 a tachycardia and providing anti-tachycardia pacing ATP, by means of the implantable pacing device IPD.

In addition, the method comprises signaling S3 an availability and/or unavailability of defibrillation to the implantable pacing device IPD by means of the implantable cardioverter defibrillator ICD.

The method moreover comprises enabling S4a and/or disabling S4b anti-tachycardia pacing ATP by means of the implantable pacing device IPD, in response to the signal 10 of the implantable cardioverter defibrillator ICD.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

REFERENCE SIGNS
1 implantable system
2 active housing
3 electrode lead
4 sternum
5, 6 sensing poles
7 shock coil
10 signal
16 detection unit
17 receiver unit
18 tachycardia pacing timing unit
19 control unit
20 fault detection device
22 external device
24 impedance measurement
26 communication unit
36 tachycardia detection
38 ventricular fibrillation
40 shock capacitors
42 defibrillation shock
H heart
ATP anti-tachycardia pacing
ICD implantable cardioverter defibrillator
IPD implantable pacing device
S1-S4b method steps

1. Implantable system for providing anti-tachycardia and/or shock therapy, comprising an implantable pacing device, in particular an implantable leadless pacemaker, and an implantable cardioverter defibrillator, in particular a non-transvenous implantable cardioverter defibrillator, wherein the implantable pacing device is configured to detect a tachycardia and to provide anti-tachycardia pacing, wherein the implantable cardioverter defibrillator is further configured to signal an availability and/or unavailability of defibrillation to the implantable pacing device, and wherein the implantable pacing device is configured, in response to the signal of the implantable cardioverter defibrillator to enable and/or disable anti-tachycardia pacing.

2. Implantable system of claim 1, wherein the implantable pacing device is configured to stimulate at least one ventricle of a human or animal heart, and wherein the implantable pacing device comprises a configurable tachyarrhythmia detection unit configured to detect a tachycardia and further comprises an anti-tachycardia pacing timing unit configured to deliver an anti-tachycardia pacing sequence in response to tachycardia detection.

3. Implantable system of claim 1, wherein the implantable pacing device comprises a receiver unit configured to receive a signal indicating an availability or unavailability of defibrillation of the implantable cardioverter defibrillator, and wherein the implantable pacing device comprises a control unit configured to enable anti-tachycardia pacing if it is detected by the receiver unit that defibrillation by the implantable cardioverter defibrillator is available and to disable anti-tachycardia pacing if it is detected by the receiver unit that defibrillation by the implantable cardioverter defibrillator is unavailable.

4. Implantable system of claim 3, wherein the receiver unit is configured to evaluate the signal indicating the availability or unavailability of defibrillation by the implantable cardioverter defibrillator, and wherein the implantable pacing device; is configured to perform a painless electrode impedance measurement of the implantable cardioverter defibrillator if it is determined that defibrillation by the implantable cardioverter defibrillator is available.

5. Implantable system of wherein the implantable cardioverter defibrillator comprises a fault detection device configured to detect the unavailability of defibrillation, in particular due to a battery exhaustion and/or fault conditions of the implantable cardioverter defibrillator.

6. Implantable system of wherein the implantable cardioverter defibrillator is configured to signal the implantable pacing device by means of intra-body-communication, in particular by means of electrical pulses, communication and/or radio signals.

7. Implantable system of claim 1, wherein the implantable pacing device prompts the cardioverter defibrillator to signal the availability of defibrillation by means of intra-body-communication before delivering anti-tachycardia pacing.

8. Implantable system of claim 1, wherein an external device, in particular a programmer and/or a wireless communication device, connected to the cardioverter defibrillator is configured to signal the availability and/or unavailability of defibrillation to the implantable pacing device.

9. Implantable system of claim 1, wherein the cardioverter defibrillator is configured to communicate a remaining operating time to the implantable pacing device, and wherein the implantable pacing device is configured to deactivate an anti-tachycardia pacing function after expiry, at expiry or a predetermined time period before expiry of an expected defibrillator operating time.

10. Implantable system of claim 1, wherein the cardioverter defibrillator is configured to signal the availability of defibrillation by performing a painless impedance measurement of its electrode system only when the cardioverter defibrillator is in an activated state.

11. Implantable system of claim 1, wherein the cardioverter defibrillator is configured to signal the availability of defibrillation by emitting a signaling pattern only when the cardioverter defibrillator is in an activated state and the cardioverter defibrillator detects tachycardia.

12. Implantable system of claim 1, wherein the cardioverter defibrillator comprises a communication unit for intrabody communication configured to signal the availability of defibrillation to the implantable pacing device.

13. Computer implemented method for providing anti-tachycardia and/or shock therapy, comprising the steps of:
providing an implantable system for providing anti-tachycardia and/or shock therapy, comprising an implantable pacing device, in particular an implantable leadless pacemaker, and an implantable cardioverter defibrillator, in particular a non-transvenous implantable cardioverter defibrillator;
detecting a tachycardia and providing anti-tachycardia pacing by means of the implantable pacing device;
signaling an availability and/or unavailability of defibrillation to the implantable pacing device by means of the implantable cardioverter defibrillator; and
enabling and/or disabling anti-tachycardia pacing by means of the implantable pacing device, in response to the signal of the implantable cardioverter defibrillator.

14. Computer program with program code to perform the method of claim 13 when the computer program is executed on a computer.

15. Computer-readable data carrier containing program code of a computer program for performing the method of claim 13 when the computer program is executed on a computer.
